(19) United States
(12) Patent Application Publication
(10) Pub. No.: US 2022/0409587 A1
(43) Pub. Date: Dec. 29, 2022

(54) METHODS OF TREATING HIV-1 INFECTION

(71) Applicant: BIOTRON LIMITED, Sydney (AU)

(72) Inventors: Carolyn Luscombe, Sydney (AU); Gary Ewart, Sydney (AU); Michelle Miller, Sydney (AU)

(21) Appl. No.: 17/779,067

(22) PCT Filed: Nov. 25, 2020
(86) PCT No.: PCT/AU20/51273

§ 371 Date: May 23, 2022

(30) Foreign Application Priority Data
Nov. 26, 2019 (AU) 2019904453
Jul. 3, 2020 (AU) 2020902273

(51) Int. Cl. A61K 31/415; A61K 31/513; A61K 31/536; A61K 31/675; A61K 31/683; A61P 31/18

(52) U.S. Cl. CPC A61K 31/415; A61K 31/513; A61K 31/536; A61K 31/675; A61K 31/683; A61P 31/18; A61K 2300/00

(57) ABSTRACT

Current antiretroviral therapy (ART) is a combination of 2-3 antiretroviral agents that has been successful in reducing HIV-1 RNA in the blood, and has improved the morbidity and mortality of HIV-1 infection and AIDS. Despite potent ART, eradication of HIV-1 infection remains elusive and there is potential for persistent virus replication in viral reservoirs that may continue to drive the pathogenic disease progression. Accordingly, there is a need for agents that assist in eradicating HIV-1 infection. The present invention relates to treating HIV-1 infection by administering N-carbamimidoyl-5-(1-methylpyrazol-4-yl)naphthalene-2-carboxamide in combination with antiretroviral agents.

### PRIORITY

The present application claims priority from Australian Provisional Patent Application No. 2019904453 (filed 26 Nov. 2019) and from Australian Provisional Patent Application No. 2020902273 (filed 3 Jul. 2020), the contents of which are incorporated in their entirety herein

### FIELD OF THE INVENTION

The present invention relates to methods of treating HIV-1 infection. In particular, the present invention relates to treating HIV-1 infection by administering N-carbamimidoyl-5-(1-methylpyrazol-4-yl)naphthalene-2-carboxamide in combination with one or more antiretroviral agents. However, it will be appreciated that the invention is not limited to this particular field of use.

### BACKGROUND OF THE INVENTION

Any discussion of the prior art throughout the specification should in no way be considered as an admission that such prior art is widely known or forms part of common general knowledge in the field.

Current antiretroviral therapy (ART) is a combination of 2-3 antiretroviral agents that has been successful in reducing HIV-1 RNA in the blood to undetectable levels (<15 copies/mL) and has improved the morbidity and mortality of HIV-1 infection and AIDS. Antiretroviral therapy consists of a combination of antiretroviral agents with at least two different modes of action against HIV-1 replication from 6 broad classes: nucleoside-analogue reverse transcriptase inhibitors (NRTIs), non-nucleoside reverse transcriptase inhibitors (NNRTIs), integrase strand transfer inhibitors (INSTI), protease inhibitors (Pls), fusion inhibitors, and entry inhibitors (Arts & Hazuda, Cold Spring Harb Perspect Med 2012, 2: a007161].

Despite potent ART, eradication of HIV-1 infection remains elusive. To date, ART has not cured an individual with HIV-1 infection (Arts & Hazuda, Cold Spring Harb Perspect Med 2012, 2: a007161; Pitman et al., Lancet HIV 2018, 5(6): 2317-e328). Sanctuary sites or reservoirs are havens for virus latency and low-level viral replication, and the current prospect for HIV-1 management is a lifetime of antiretroviral therapy. Without HIV-1 eradication there is potential for persistent virus replication in viral reservoirs that may continue to drive the pathogenic disease progression (Pierson at al., Annu Rev Immunol 2000, 18: 665-708; Honeycutt et al., J Clin Invest 2016, 126: 1353-66). Antiretroviral therapy advances to date have predominantly focused on agents that affect replication of the viral genome and not agents that eliminate HIV-1 infection.

Consequently, there is a need for agents that assist in eradicating HIV-1 infection.

It is an object of the present invention to overcome or ameliorate at least one of the disadvantages of the prior art, or to provide a useful alternative.

### SUMMARY OF THE INVENTION

The present invention relates to the surprising finding that administering BIT225 in combination with antiretroviral agents treats HIV-1 infection and modulates the immune system in a subject. Such action may assist in eradicating reservoirs of HIV-1 infection that remain despite effective ART treatment. In addition, such action may result in improvement in inflammatory-based adverse health outcomes that remain despite effective ART treatment.

The chemical structure of BIT225 is shown below:

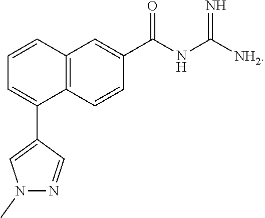 [SMILES: Cn1cc(-c2cccc3cc(C(=O)NC(=N)N)ccc23)cn1]

In one embodiment, the present invention relates to a method for treating HIV-1 infection and regulating immune system function by lessening systemic inflammation and augmenting immune activation in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and regulating immune system function by lessening systemic inflammation and augmenting immune activation in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection and regulating immune system function by lessening systemic inflammation and augmenting immune activation in a subject.

In one embodiment, the present invention relates to a method for treating HIV-1 infection and altering HIV-induced immune system dysregulation by lessening systemic inflammation and augmenting immune activation in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and altering HIV-induced immune system dysregulation by lessening systemic inflammation and augmenting immune activation in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use treating HIV-1 infection and altering HIV-induced immune system dysregulation by lessening systemic inflammation and augmenting immune activation in a subject.

In one embodiment, the immune system is the innate immune system.

In one embodiment, the systemic inflammation is myeloid and/or monocyte inflammation.

In one embodiment, the present invention relates to a method for treating HIV-1 infection and modulating the immune system in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and modulating the immune system in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection and modulating the immune system in a subject.

In one embodiment, the present invention relates to a method for treating HIV-1 infection and modulating the innate immune system in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and modulating the innate immune system in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection and modulating the innate immune system in a subject.

In one embodiment, the present invention relates to a method for treating HIV-1 infection and activating the immune system in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and activating the immune system in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection and activating the immune system in a subject.

In one embodiment, the present invention relates to a method for treating HIV-1 infection and activating the innate immune system in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and activating the innate immune system in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection and activating the innate immune system in a subject.

In one embodiment, the present invention relates to a method for treating HIV-1 infection and stimulating the immune system in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and stimulating the immune system in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection and stimulating the immune system in a subject.

In one embodiment, the present invention relates to a method for treating HIV-1 infection and stimulating the innate immune system in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and stimulating the innate immune system in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection and stimulating the innate immune system in a subject.

In one embodiment, the present invention relates to a method for treating HIV-1 infection and unmasking HIV-1 infected cells in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and unmasking HIV-1 infected cells in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection and unmasking HIV-1 infected cells in a subject.

In one embodiment, the present invention relates to a method for treating HIV-1 infection and eradicating HIV-1 reservoirs in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and eradicating HIV-1 reservoirs in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection and eradicating HIV-1 reservoirs in a subject.

In one embodiment, the present invention relates to a method for treating HIV-1 infection in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

In one embodiment, the present invention provides use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection in a subject.

In one embodiment, the present invention provides a combination comprising a) one or more antiretroviral agents and b)N-carbamimidoyl-5-(1-methylpyrazol-4-yl)naphthalene-2-carboxamide or a pharmaceutically acceptable salt thereof, for use in treating HIV-1 infection in a subject.

In one embodiment, administration of the combination or medicament regulates immune system function by lessening systemic inflammation and augmenting immune activation.

In one embodiment, administration of the combination or medicament alters HIV-induced immune system dysregulation by lessening systemic inflammation and augmenting immune activation.

In one embodiment, the immune system is the innate immune system.

In one embodiment, the systemic inflammation is myeloid and/or monocyte inflammation.

In one embodiment, administration of the combination or medicament modulates the immune system of the subject.

In one embodiment, administration of the combination or medicament modulates the innate immune system of the subject.

In one embodiment, administration of the combination or medicament stimulates the immune system of the subject.

In one embodiment, administration of the combination or medicament stimulates the innate immune system of the subject.

In one embodiment, administration of the combination or medicament activates the immune system of the subject.

In one embodiment, administration of the combination or medicament activates the innate immune system of the subject.

In one embodiment, administration of the combination or medicament unmasks HIV-1 infected cells in the subject.

In one embodiment, administration of the combination or medicament increases the number of CD4⁺ T cells compared to administration of the one or more antiretroviral agents alone.

In one embodiment, administration of the combination or medicament reverses HIV-1-related defects in CD4⁺ T cell signalling.

In one embodiment, administration of the combination or medicament reverses HIV-1-related defects in CD4⁺ T cell signalling compared to administration of the one or more antiretroviral agents alone.

In one embodiment, administration of the combination or medicament increases the number of CD8⁺ T cells compared to administration of the one or more antiretroviral agents alone.

In one embodiment, administration of the combination or medicament reverses HIV-1-related defects in CD8⁺ T cell signalling.

In one embodiment, administration of the combination or medicament reverses HIV-1-related defects in CD8⁺ T cell signalling compared to administration of the one or more antiretroviral agents alone.

In one embodiment, administration of the combination or medicament reverses the down modulating effects of Vpu on cellular receptors.

In one embodiment, administration of the combination or medicament increases the number of NK cells compared to administration of the one or more antiretroviral agents alone.

In one embodiment, administration of the combination or medicament reverses HIV-1-related defects in NK signalling.

In one embodiment, administration of the combination or medicament reverses HIV-1-related defects in NK signalling compared to administration of the one or more antiretroviral agents alone.

In one embodiment, administration of the combination or medicament reverses the down modulating effects of Vpu in NK cells.

In one embodiment, administration of the combination or medicament reverses the down modulating effects of Vpu in NK cells compared to administration of the one or more antiretroviral agents alone.

In one embodiment, administration of the combination or medicament increases key cell surface receptors for efficient NK signalling and degranulation.

In one embodiment, administration of the combination or medicament increases cell surface receptors required for efficient NK signalling and degranulation compared to administration of the one or more antiretroviral agents alone.

In one embodiment, administration of the combination or medicament reduces the level of sCD163 in plasma compared to administration of the one or more antiretroviral agents alone.

In one embodiment, administration of the combination or medicament reduces activation of monocytes and/or macrophages compared to administration of the one or more antiretroviral agents alone.

In one embodiment, administration of the combination or medicament increases the level of IL-21 in plasma compared to administration of the one or more antiretroviral agents alone.

In one embodiment, administration of the combination or medicament increases the number of T Helper 17 cells (Th17 cells) compared to administration of the one or more antiretroviral agents alone.

In one embodiment, administration of the combination or medicament restores the function of Th17 cells compared to administration of the one or more antiretroviral agents alone.

In one embodiment, administration of the combination or medicament increases the number of follicular helper CD4 T cells (Tfh cells) compared to administration of the one or more antiretroviral agents alone.

In one embodiment, administration of the combination or medicament modulates HIV-1 specific antibody responses.

In one embodiment, administration of the combination or medicament reduces downregulation of CD28 expression on CD4⁺ T cells infected with HIV-1 compared to administration of the one or more antiretroviral agents alone.

In one embodiment, administration of the combination or medicament reduces downregulation of CCR7 expression on CD4⁺ T cells infected with HIV-1 compared to administration of the one or more antiretroviral agents alone.

In one embodiment, administration of the combination or medicament reduces downregulation of CD80 expression on monocyte-derived macrophages infected with HIV-1 compared to administration of the one or more antiretroviral agents alone.

In one embodiment, administration of the combination or medicament reduces downregulation of CD86 expression on monocyte-derived macrophages infected with HIV-1 compared to administration of the one or more antiretroviral agents alone.

In one embodiment, administration of the combination or medicament enhances co-stimulatory signals required for T cell activation and homing compared to administration of the one or more antiretroviral agents alone.

In one embodiment, administration of the combination or medicament enhances immune surveillance of HIV-1 compared to administration of the one or more antiretroviral agents alone.

In one embodiment, administration of the combination or medicament enhances immune surveillance of HIV-1 compared to administration of the one or more antiretroviral agents alone.

In one embodiment, the one or more antiretroviral agents comprise a non-nucleoside reverse transcriptase inhibitor (NNRTI).

In one embodiment, the one or more antiretroviral agents comprise a nucleoside reverse transcriptase inhibitor (NRTI).

In one embodiment, the one or more antiretroviral agents comprise two NRTIs.

In one embodiment, the one or more antiretroviral agents comprise a NNRTI and a NRTI.

In one embodiment, the one or more antiretroviral agents comprise a NNRTI and two NRTIs.

In one embodiment, the NNRTI is efavirenz.

In one embodiment, NRTI is emtricitabine.

In one embodiment, the NRTI is tenofovir disoproxil fumarate (tenofovir DF).

In one embodiment, the one or more antiretroviral agents comprise efavirenz, emtricitabine and tenofovir DF (i.e., efavirenz/emtricitabine/tenofovir DF).

In one embodiment, the one or more antiretroviral agents consist of efavirenz, emtricitabine and tenofovir DF (efavirenz/emtricitabine/tenofovir DF).

In one embodiment, the efavirenz is administered to the subject at a dosage of 600 mg.

In one embodiment, the emtricitabine is administered to the subject at a dosage of 200 mg.

In one embodiment, the tenofovir DF is administered to the subject at a dosage of 300 mg.

In one embodiment, the present invention relates to a method for regulating immune function by lessening systemic inflammation and augmenting immune activation in a subject infected with HIV-1, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for regulating immune function by lessening systemic inflammation and augmenting immune activation in a subject infected with HIV-1.

In one embodiment, the present invention provides N-carbamimidoyl-5-(1-methylpyrazol-4-yl)naphthalene-2-carboxamide or a pharmaceutically acceptable salt thereof, for use in regulating immune function by lessening systemic inflammation and augmenting immune activation in a subject infected with HIV-1.

In one embodiment, the present invention relates to a method for altering HIV-induced immune dysregulation by lessening systemic inflammation and augmenting immune activation in a subject infected with HIV-1, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for altering HIV-induced immune dysregulation by lessening systemic inflammation and augmenting immune activation in a subject infected with HIV-1.

In one embodiment, the present invention provides N-carbamimidoyl-5-(1-methylpyrazol-4-yl)naphthalene-2-carboxamide or a pharmaceutically acceptable salt thereof, for use in altering HIV-induced immune dysregulation by lessening systemic inflammation and augmenting immune activation in a subject infected with HIV-1.In one embodiment, the immune system is the innate immune system.

In one embodiment, the systemic inflammation is myeloid and/or monocyte inflammation.

In one embodiment, the present invention relates to a method for modulating the immune system in a subject infected with HIV-1, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for modulating the immune system in a subject infected with HIV-1.

In one embodiment, the present invention provides N-carbamimidoyl-5-(1-methylpyrazol-4-yl)naphthalene-2-carboxamide or a pharmaceutically acceptable salt thereof, for use in modulating the immune system in a subject infected with HIV-1.

In one embodiment, the present invention relates to a method for modulating the innate immune system in a subject infected with HIV-1, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for modulating the innate immune system in a subject infected with HIV-1.

In one embodiment, the present invention provides N-carbamimidoyl-5-(1-methylpyrazol-4-yl)naphthalene-2-carboxamide or a pharmaceutically acceptable salt thereof, for use in modulating the innate immune system in a subject infected with HIV-1.

In one embodiment, the present invention relates to a method for activating the immune system in a subject infected with HIV-1, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for activating the immune system in a subject infected with HIV-1.

In one embodiment, the present invention provides N-carbamimidoyl-5-(1-methylpyrazol-4-yl)naphthalene-2-carboxamide or a pharmaceutically acceptable salt thereof, for use in activating the immune system in a subject infected with HIV-1.

In one embodiment, the present invention relates to a method for activating the innate immune system in a subject infected with HIV-1, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for activating the immune system in a subject infected with HIV-1.

In one embodiment, the present invention provides N-carbamimidoyl-5-(1-methylpyrazol-4-yl)naphthalene-2-carboxamide or a pharmaceutically acceptable salt thereof, for use in activating the immune system in a subject infected with HIV-1.

In one embodiment, the present invention relates to a method for stimulating the immune system in a subject infected with HIV-1, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for stimulating the immune system in a subject infected with HIV-1.

In one embodiment, the present invention provides N-carbamimidoyl-5-(1-methylpyrazol-4-yl)naphthalene-2-carboxamide or a pharmaceutically acceptable salt thereof, for use in stimulating the immune system in a subject infected with HIV-1.

In one embodiment, the present invention relates to a method for stimulating the innate immune system in a subject infected with HIV-1, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for stimulating the innate immune system in a subject infected with HIV-1.

In one embodiment, the present invention provides N-carbamimidoyl-5-(1-methylpyrazol-4-yl)naphthalene-2-carboxamide or a pharmaceutically acceptable salt thereof, for use in stimulating the innate immune system in a subject infected with HIV-1.

In one embodiment, the present invention provides a method for reducing downregulation of CD28 expression on CD4⁺ T cells infected with HIV-1 in a subject, comprising administering N-carbamimidoyl-5-(1-methylpyrazol-4-yl)naphthalene-2-carboxamide or a pharmaceutically acceptable salt thereof to the subject.

In one embodiment, the present invention provides a method for reducing downregulation of CD28 expression on CD4⁺ T cells infected with HIV-1, comprising administering N-carbamimidoyl-5-(1-methylpyrazol-4-yl)naphthalene-2-carboxamide or a pharmaceutically acceptable salt thereof to the T cells.

In one embodiment, the present invention provides use of N-carbamimidoyl-5-(1-methylpyrazol-4-yl)naphthalene-2-carboxamide or a pharmaceutically acceptable salt thereof for reducing downregulation of CD28 expression on CD4⁺ T cells infected with HIV-1.

In one embodiment, the present invention provides N-carbamimidoyl-5-(1-methylpyrazol-4-yl)naphthalene-2-carboxamide or a pharmaceutically acceptable salt thereof for use in reducing downregulation of CD28 expression on CD4⁺ T cells infected with HIV-1.

In one embodiment, the effect of N-carbamimidoyl-5-(1-methylpyrazol-4-yl)naphthalene-2-carboxamide or a pharmaceutically acceptable salt thereof on CD28 expression on CD4⁺ T cells infected with HIV-1 is dependent on Vpu expression on the T cells.

In one embodiment, the present invention provides a method for reducing downregulation of CCR7 expression on CD4⁺ T cells infected with HIV-1 in a subject, comprising administering N-carbamimidoyl-5-(1-methylpyrazol-4-yl)naphthalene-2-carboxamide or a pharmaceutically acceptable salt thereof to the subject.

In one embodiment, the present invention provides a method for reducing downregulation of CCR7 expression on CD4⁺ T cells infected with HIV-1, comprising administering N-carbamimidoyl-5-(1-methylpyrazol-4-yl)naphthalene-2-carboxamide or a pharmaceutically acceptable salt thereof to the T cells.

In one embodiment, the present invention provides use of N-carbamimidoyl-5-(1-methylpyrazol-4-yl)naphthalene-2-carboxamide or a pharmaceutically acceptable salt thereof for reducing downregulation of CCR7 expression on CD4⁺ T cells infected with HIV-1.

In one embodiment, the present invention provides N-carbamimidoyl-5-(1-methylpyrazol-4-yl)naphthalene-2-carboxamide or a pharmaceutically acceptable salt thereof for use in reducing downregulation of CCR7 expression on CD4⁺ T cells infected with HIV-1.

In one embodiment, the effect of N-carbamimidoyl-5-(1-methylpyrazol-4-yl)naphthalene-2-carboxamide or a pharmaceutically acceptable salt thereof on CCR7 expression on CD4⁺ T cells infected with HIV-1 is dependent on Vpu expression on the T cells.

In one embodiment, the present invention provides a method for reducing downregulation of CD80 expression on monocyte-derived macrophages infected with HIV-1 in a subject, comprising administering N-carbamimidoyl-5-(1-methylpyrazol-4-yl)naphthalene-2-carboxamide or a pharmaceutically acceptable salt thereof to the subject.

In one embodiment, the present invention provides a method for reducing downregulation of CD80 expression on monocyte-derived macrophages infected with HIV-1, comprising administering N-carbamimidoyl-5-(1-methylpyrazol-4-yl)naphthalene-2-carboxamide or a pharmaceutically acceptable salt thereof to the monocyte-derived macrophages.

In one embodiment, the present invention provides use of N-carbamimidoyl-5-(1-methylpyrazol-4-yl)naphthalene-2-carboxamide or a pharmaceutically acceptable salt thereof for reducing downregulation of CD80 expression on monocyte-derived macrophages infected with HIV-1.

In one embodiment, the present invention provides N-carbamimidoyl-5-(1-methylpyrazol-4-yl)naphthalene-2-carboxamide or a pharmaceutically acceptable salt thereof for use in reducing downregulation of CD80 expression on monocyte-derived macrophages infected with HIV-1.

In one embodiment, the effect of N-carbamimidoyl-5-(1-methylpyrazol-4-yl)naphthalene-2-carboxamide or a pharmaceutically acceptable salt thereof on CD80 expression on monocyte-derived macrophages infected with HIV-1 is dependent on Vpu expression on the monocyte-derived macrophages.

In one embodiment, the present invention provides a method for reducing downregulation of CD86 expression on monocyte-derived macrophages infected with HIV-1 in a subject, comprising administering N-carbamimidoyl-5-(1-methylpyrazol-4-yl)naphthalene-2-carboxamide or a pharmaceutically acceptable salt thereof to the subject.

In one embodiment, the present invention provides a method for reducing downregulation of CD86 expression on monocyte-derived macrophages infected with HIV-1, comprising administering N-carbamimidoyl-5-(1-methylpyrazol-4-yl)naphthalene-2-carboxamide or a pharmaceutically acceptable salt thereof to the monocyte-derived macrophages.

In one embodiment, the present invention provides use of N-carbamimidoyl-5-(1-methylpyrazol-4-yl)naphthalene-2-carboxamide or a pharmaceutically acceptable salt thereof for reducing downregulation of CD86 expression on monocyte-derived macrophages infected with HIV-1.

In one embodiment, the present invention provides N-carbamimidoyl-5-(1-methylpyrazol-4-yl)naphthalene-2-carboxamide or a pharmaceutically acceptable salt thereof for use in reducing downregulation of CD86 expression on monocyte-derived macrophages infected with HIV-1.

In one embodiment, the effect of N-carbamimidoyl-5-(1-methylpyrazol-4-yl)naphthalene-2-carboxamide or a pharmaceutically acceptable salt thereof on CD86 expression on monocyte-derived macrophages infected with HIV-1 is not dependent on Vpu expression on the monocyte-derived macrophages.

In one embodiment, the present invention provides a method for enhancing co-stimulatory signals required for T cell activation and homing in a subject infected with HIV-1, comprising administering N-carbamimidoyl-5-(1-methylpyrazol-4-yl)naphthalene-2-carboxamide or a pharmaceutically acceptable salt thereof to the subject.

In one embodiment, the present invention provides use of N-carbamimidoyl-5-(1-methylpyrazol-4-yl)naphthalene-2-carboxamide or a pharmaceutically acceptable salt thereof for enhancing co-stimulatory signals required for T cell activation and homing in a subject infected with HIV-1.

In one embodiment, the present invention provides N-carbamimidoyl-5-(1-methylpyrazol-4-yl)naphthalene-2-carboxamide or a pharmaceutically acceptable salt thereof for use in for enhancing co-stimulatory signals required for T cell activation and homing in a subject infected with HIV-1.

In one embodiment, the present invention provides a method for enhancing immune surveillance of HIV-1 in a subject, comprising administering N-carbamimidoyl-5-(1-methylpyrazol-4-yl)naphthalene-2-carboxamide or a pharmaceutically acceptable salt thereof to the subject.

In one embodiment, the present invention provides use of N-carbamimidoyl-5-(1-methylpyrazol-4-yl)naphthalene-2-carboxamide or a pharmaceutically acceptable salt thereof for enhancing immune surveillance of HIV-1.

In one embodiment, the present invention provides N-carbamimidoyl-5-(1-methylpyrazol-4-yl)naphthalene-2-carboxamide or a pharmaceutically acceptable salt thereof for use in enhancing immune surveillance of HIV-1.

In one embodiment, the present invention relates to a method for unmasking HIV-1 infected cells in a subject, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for unmasking HIV-1 infected cells in a subject.

In one embodiment, the present invention provides N-carbamimidoyl-5-(1-methylpyrazol-4-yl)naphthalene-2-carboxamide or a pharmaceutically acceptable salt thereof, for use in unmasking HIV-1 infected cells in a subject.

In one embodiment, the present invention relates to a method for eradicating HIV-1 reservoirs in a subject, comprising administering to the subject N-carbamimidoyl-5-(1-methylpyrazol-4-yl)naphthalene-2-carboxamide or a pharmaceutically acceptable salt thereof.

In one embodiment, the present invention provides use of N-carbamimidoyl-5-(1-methylpyrazol-4-yl)naphthalene-2-carboxamide or a pharmaceutically acceptable salt thereof, for the manufacture of a medicament for eradicating HIV-1 reservoirs in a subject.

In one embodiment, the present invention provides N-carbamimidoyl-5-(1-methylpyrazol-4-yl)naphthalene-2-carboxamide or a pharmaceutically acceptable salt thereof, for use in eradicating HIV-1 reservoirs in a subject.

In one embodiment, the N-carbamimidoyl-5-(1-methyl-1 H-pyrazol-4-yl)-2-naphthamide or a pharmaceutically acceptable salt thereof is administered to the subject by a route selected from oral, nasal, intravenous, intraperitoneal, inhalation and topical.

In one embodiment, the N-carbamimidoyl-5-(1-methyl-1 H-pyrazol-4-yl)-2-naphthamide or a pharmaceutically acceptable salt thereof is administered to the subject orally.

In one embodiment, the N-carbamimidoyl-5-(1-methyl-1 H-pyrazol-4-yl)-2-naphthamide or a pharmaceutically acceptable salt thereof is administered to the subject daily.

In one embodiment, the N-carbamimidoyl-5-(1-methyl-1 H-pyrazol-4-yl)-2-naphthamide or a pharmaceutically acceptable salt thereof is administered to the subject twice daily.

In one embodiment, the N-carbamimidoyl-5-(1-methyl-1 H-pyrazol-4-yl)-2-naphthamide or a pharmaceutically acceptable salt thereof is administered to the subject at a dosage of about 100 mg to about 600 mg.

In one embodiment, the N-carbamimidoyl-5-(1-methyl-1 H-pyrazol-4-yl)-2-naphthamide or a pharmaceutically acceptable salt thereof is administered to the subject orally and at a dosage of about 600 mg.

In one embodiment, the N-carbamimidoyl-5-(1-methyl-1 H-pyrazol-4-yl)-2-naphthamide or a pharmaceutically acceptable salt thereof is administered to the subject orally and at a dosage of about 200 mg.

In one embodiment, the N-carbamimidoyl-5-(1-methyl-1 H-pyrazol-4-yl)-2-naphthamide or a pharmaceutically acceptable salt thereof is administered to the subject orally and at a dosage of about 100 mg.

In one embodiment, the N-carbamimidoyl-5-(1-methyl-1 H-pyrazol-4-yl)-2-naphthamide or a pharmaceutically acceptable salt thereof is administered to the subject orally and daily.

In one embodiment, the N-carbamimidoyl-5-(1-methyl-1 H-pyrazol-4-yl)-2-naphthamide or a pharmaceutically acceptable salt thereof is administered to the subject orally and twice daily.

In one embodiment, the N-carbamimidoyl-5-(1-methyl-1 H-pyrazol-4-yl)-2-naphthamide or a pharmaceutically acceptable salt thereof is administered orally, once daily at a dosage of about 200 mg.

In one embodiment, the N-carbamimidoyl-5-(1-methyl-1 H-pyrazol-4-yl)-2-naphthamide or a pharmaceutically acceptable salt thereof is administered orally, twice daily at a dosage of about 200 mg.

In one embodiment, the N-carbamimidoyl-5-(1-methyl-1 H-pyrazol-4-yl)-2-naphthamide or a pharmaceutically acceptable salt thereof is administered orally, once daily at a dosage of about 100 mg.

In one embodiment, the N-carbamimidoyl-5-(1-methyl-1 H-pyrazol-4-yl)-2-naphthamide or a pharmaceutically acceptable salt thereof is administered orally, twice daily at a dosage of about 100 mg.

### BRIEF DESCRIPTION OF THE DRAWINGS

FIG. 1—Group mean change from baseline of activated CD4⁺ T cells (CD4^{+/}HLA-DR^{+/}CD38⁺) over the 12-week treatment period with 200 mg BIT225 QD (circles) or placebo (squares) and ART. There was a statistically significant (P<0.01, linear model) sustained delay in decline of CD4⁺ activated T-cell numbers during the BIT225 treatment period, compared to placebo.

FIG. 2—Group mean change from baseline of activated CD8⁺ cell (CD8⁺/HLA-DR⁺/CD38⁺) numbers during 12 Weeks of 200 mg BIT225 QD (circles) or placebo (squares) treatment with ART. The linear model for change from baseline as a function of Day and Treatment group showed that, after controlling for days of treatment, the BIT225 cohort had a smaller decrease in activated CD8⁺ T-cells during the BIT225 treatment period: Estimated average difference 85 cells/ml (±29, SEM), which was statistically significant (P<0.01).

FIG. 3—Group mean change from baseline of NK cell (CD8⁺/HLA-DR⁺/CD38⁺) numbers during 12 Weeks of 200 mg BIT225 QD (circles) or placebo (squares) treatment with ART. The linear model for change from baseline as a function of Day and Treatment group showed that, after controlling for days of treatment, the BIT225 cohort had a smaller decrease in NK cells during the BIT225 treatment period: Estimated average difference 71 cells/ml (±23, SEM), which was statistically significant (P<0.01).

FIG. 4—Time course of mean soluble CD163 (sCD163) ng/mL change from baseline during 12 weeks of treatment with ART plus 200 mg BIT225 QD (circles) or placebo (diamonds). Two-way ANOVA for BIT225 versus Placebo, controlling for day of treatment, was done using R statistical software by fitting the linear model: β0+β1.day+β1.I_{RX}., where I_{RX} takes the values 0 (for BIT225 treatment), or 1 (for Placebo). The estimate; β1=208±99 (SE) ng/ml indicates a statistically larger overall decrease in sCD163 for the BIT225 treated group (P=0.036). The difference between placebo and control at Day 7 is statistically significant by Welch's T-test (P=0.045).

FIG. 5—Time course of plasma IL-21 (ng/mL) production during 12 Weeks of treatment with 200 mg BIT225 QD (triangles) or placebo (circles) and ART. ANOVA analysis found that the difference between BIT225 and placebo cohorts over the first 3 weeks was statistically significant (P=0.02).

FIG. 6—Plasma Membrane expression of CD28 on CD4⁺ T cells treated with 3 μM of BIT225 at 72 hours post-infection with HIV-1 NL4-3 or mutants deficient in Vpu, Nef or both Vpu and Nef. *** denotes P<0.001; n/s denotes P>0.05.

FIG. 7—Plasma Membrane expression of CD80 on MDM treated with 3 μM of BIT225 at 72 hours post-infection with HIV-1NL4-3 or mutants deficient in Vpu, Nef or both Vpu and Nef. ** denotes P<0.01.

FIG. 8—Plasma Membrane expression of CD86 on MDM treated with 3 μM of BIT225 at 72 hours post-infection with HIV-1 NL4-3 or mutants deficient in Vpu, Nef or both Vpu and Nef. *** denotes P<0.001.

FIG. 9—Plasma Membrane expression of CCR7 on CD4⁺ T cells treated with 3 μM of BIT225 at 72 hours post-infection with HIV-1 NL4-3 or mutants deficient in Vpu, Nef or both Vpu and Nef. ** denotes P<0.01; * denotes P<0.05; n/s denotes P>0.05

### DEFINITIONS

In describing and claiming the present invention, the following terminology has been used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

In the context of the invention the term “subject” includes any human or non-human animal. The term “non-human animal” includes all vertebrates, for example mammals and non-mammals, such as non-human primates, horses, cows, dogs, etc.

In the context of the present invention, the words “comprise”, “comprising” and the like are to be construed in their inclusive, as opposed to their exclusive, sense, that is in the sense of “including, but not limited to”.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Other than in the operating examples, or where otherwise indicated, all numbers expressing quantities of ingredients or reaction conditions used herein are to be understood as modified in all instances by the term “about”.

The recitation of a numerical range using endpoints includes all numbers subsumed within that range (e.g., 1 to 5 includes 1, 1.5, 2, 2.75, 3, 3.80, 4, 5, etc.).

As used herein, the term “administration of a combination” means administration of two or more agents to a subject of interest as part of a single therapeutic regimen. The administration(s) can be either simultaneous or sequential, i.e., administering one agent followed by administering of a second (and/or a third one, etc.) at a later time, as long as the agents administered co-exist in the subject being treated, or at least one agent will have the opportunity to act upon the same target tissues of other agents while said target tissues are still under the influence of said other agents. In a certain embodiment, agents to be administered can be included in a single pharmaceutical composition and administered together. In a certain embodiment, the agents are administered simultaneously, including through separate routes. In a certain embodiment, one or more agents are administered continuously, while other agents are administered only at predetermined intervals (such as a single large dosage, or twice a week at smaller dosages, etc.).

The present invention includes within its scope pharmaceutically acceptable salts. Agents of the present invention may in some cases form salts, which are also within the scope of this invention. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the R substituents comprise an acid moiety such as a carboxyl group). Also included herein are quaternary ammonium salts such as alkylammonium salts. Pharmaceutically acceptable (i.e., non-toxic, physiologically acceptable) salts are preferred, although other salts are useful, for example, in isolation or purification steps which may be employed during preparation. Salts of the agents may be formed, for example, by reacting a compound with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxy ethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like.

Exemplary basic salts (formed, for example, wherein the substituent comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like.

The basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the agents of the invention are also contemplated herein.

As used herein, the term “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The present invention further provides pharmaceutical compositions comprising the agents of the invention as active ingredients along with pharmaceutically acceptable additives/excipients/adjuvants/vehicles.

Agents of the present invention may be used in a pharmaceutical composition, e.g., combined with a pharmaceutically acceptable carrier, for administration to a patient. Such a composition may also contain diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. Such additional factors and/or agents may be included in the pharmaceutical composition to produce a synergistic effect with agents of the invention, or to minimize side effects caused by the compound of the invention.

The pharmaceutical compositions of the invention may be in the form of a liposome or micelles in which agents of the present invention are combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The composition may be administered in a variety of ways including orally, nasally, buccally, sublingually, intravenously, transmucosally, parenterally, by inhalation, spray, transdermally, subcutaneously, intrathecally, topically or rectally and may be formulated according to methods known in the art.

The term “subject with a HIV-1 infection”, “subject infected with HIV-1”, “patient with a HIV-1 infection” or “patient infected with HIV-1” as used herein means any subject having HIV-1 infection and includes treatment-naive subjects or patients and treatment-experienced patients having the HIV-1 infection.

The term “treatment-naive subject” or “treatment-naive patient” as used herein means subjects or patients having HIV-1 who have never been treated with any anti-retroviral agents or any interferon.

The term “treatment-experienced” subjects or patients as used herein means those subjects or patients having HIV-1 who have initiated some form of anti HIV-1 therapy.

The term “antiretroviral agent” as used herein means any agent that is used in the treatment of an infectious disease caused by a virus. Suitable antiviral agents for use in the treatment of HIV-1 include, but are not limited to, reverse transcriptase inhibitors, such as nucleoside reverse transcriptase inhibitors and non-nucleoside reverse transcriptase inhibitors, protease inhibitors, and fusion inhibitors.

The terms “nucleoside reverse transcriptase inhibitor” and “NRTI” as used herein mean nucleosides and nucleotides and analogues thereof that inhibit the activity of HIV-1 reverse transcriptase, the enzyme which catalyzes the conversion of viral genomic HIV-1 RNA into proviral HIV-1 DNA.

The terms “non-nucleoside reverse transcriptase inhibitor” and “NNRTI” as used herein mean non-nucleosides that inhibit the activity of HIV-1 reverse transcriptase.

The terms “protease inhibitor” and “Pl” as used herein mean inhibitors of the HIV-1 protease, an enzyme required for the proteolytic cleavage of viral polyprotein precursors into the individual functional proteins found in infectious HIV-1. HIV-1 protease inhibitors include compounds having a peptidomimetic structure, high molecular weight (7600 daltons) and substantial peptide character, as well as nonpeptide protease inhibitors.

The term “fusion inhibitor” as used herein means agents that block the HIV-1 virus from entering human cells.

The term “regulating immune system function” means changing how the body's immune system responds to HIV-1 infection.

The term “lessening systemic inflammation” means reducing HIV-1-induced inflammation in the body to a desired level.

The term “augmenting immune activation” means improving the ability of the body's immune system to respond to HIV-1 infection.

The term “HIV-induced immune dysregulation” means the adverse changes induced on the immune system by HIV-1 virus to prevent the immune system from mounting an appropriate response.

The term “unmasking HIV-1 infected cells” as used herein means exposing HIV-1 infected cells to the immune system.

The terms “modulating”, “modulation” or “modulate” as used herein means alteration to a desired level.

The terms “activating”, “activation” or “activate” as used herein means inducing an appropriate response.

The terms “stimulating”, “stimulation” or “stimulate” as used herein means to increase activation or increase activity to a desired level.

### PREFERRED EMBODIMENT OF THE INVENTION

Although the invention has been described with reference to certain embodiments detailed herein, other embodiments can achieve the same or similar results. Variations and modifications of the invention will be obvious to those skilled in the art and the invention is intended to cover all such modifications and equivalents.

The present invention combines BIT225 with antiretroviral agents in the treatment of HIV-1 infection in a subject.

The present invention is further described by the following non-limiting examples.

### Example 1-Production of Bit225

A mixture of 5-bromo-2-naphthoic acid (2.12 g, 8.44 mmol), 1-methyl-4-(4,4,5,5-tetramethyl-1,3,2-dioxaborolan-2-yl)-1H-pyrazole (1.84 g, 8.86 mmol), and tetrakis(triphenylphosphine)palladium(0) (502 mg, 0.435 mmol) in a 250 mL round bottomed flask was evacuated and purged with nitrogen (in three cycles). Acetonitrile (40 mL) and 2M aqueous sodium carbonate (10 mL) were added to the mixture via syringe, and the mixture was heated under reflux under nitrogen for 22 hours. The reaction mixture was allowed to cool before the addition of 1 M aqueous hydrochloric acid (30 mL) and it was then extracted with ethyl acetate (3×50 mL). The combined organic layers were dried (MgSO₄), filtered, and concentrated in vacuo to provide a crude product (2.98 g after air drying). This crude material was dissolved in hot ethanol (150 mL) and filtered while hot to remove a yellow impurity (120 mg). The filtrate was concentrated in vacuo and the residue was recrystallised from dichloromethane (30 mL) to provide 5-(1-methyl-1 H-pyrazol-4-yl)-2-naphthoic acid as a white solid (724 mg, 34%). A second crop of 5-(1-methyl-1 H-pyrazol-4-yl)-2-naphthoic acid (527 mg, 25%) was obtained from the concentrated mother liquors by recrystallisation from dichloromethane (20 mL).

Oxalyl chloride (1.1 mL, 13 mmol) was added to the solution of 5-(1-methyl-1 H-pyrazol-4-yl)-2-naphthoic acid (1.19 g, 4.71 mmol) in anhydrous dichloromethane (200 mL (which was added in portions during the reaction to effect dissolution)) containing dimethylformamide (2 drops) under nitrogen and the mixture was stirred at room temperature for 4.25 hours. The reaction mixture was then heated for 1 hour at 40° C., before being concentrated under reduced pressure. The resulting crude acid chloride was suspended in anhydrous tetrahydrofuran (50 mL) and this mixture was added dropwise to a solution of guanidine hydrochloride (2.09 g, 21.9 mmol) in 2M aqueous sodium hydroxide (15 mL, 30 mmol) and the reaction mixture was then stirred for 30 minutes. The organic phase was separated, and the aqueous phase was extracted with chloroform (3×30 mL) followed by ethyl acetate (3×30 mL). The combined organic extracts were washed sequentially with 1 M aqueous sodium hydroxide (60 mL) and water (40 mL), then dried (Na₂SO₄) and concentrated in vacuo to give a glassy solid (1.45 g after drying under high vacuum). This solid was dissolved in dichloromethane which was then allowed to evaporate slowly to give BIT225 as a yellow solid (1.15 g, 83%).

### Example 2-Co-Administration of BIT225 and Art in Patients with HIV-1 Infection

A Phase 2, multi-centre, randomized, placebo-controlled, double-blind study of BIT225 in combination with ART (Atripla—efavirenz/emtricitabine/tenofovir DF) was undertaken in patients with HIV-1 infection that were treatment-naive. Patients were randomized into two groups on a 2:1 basis, with one group receiving BIT225 (100 mg QD; 6 patients: placebo; 3 patients), and one group receiving BIT225 (200 mg QD, 18 patients: placebo; 9 patients), with a total of 36 patients enrolled in the trial. The 100 mg BIT225 group was primarily included for detailed PK analyses due to limitations on the quantity of blood that could be drawn at time points coinciding with analyses of viral load decay, which were limited to the 200 mg cohort. All subjects received the standard dose of ART in addition to 12 weeks BIT225 or placebo study treatment. At the conclusion of the trial, patients remained on ART as per standard treatment protocols. HIV-1-infected subjects, males and females, aged 18 to 65 years inclusive, who were naive to ART were recruited. Individuals had HIV-1 RNA>5,000 copies/mL and CD4⁺ T cell count>100 cells/mm³.

The trial was registered with the Australian New Zealand Clinical Trials Registry (ANZCTR), UTN:U1111-1191-2194.

The study was approved by the Institutional Review Board of the Faculty of Medicine, Chulalongkorn University, Bangkok and the Research Ethics Committee of the Faculty of Medicine, Chiang Mai University, Chiang Mai, Thailand. The trial was performed at two sites in Thailand: The HIV Netherlands Australia Thailand Research Collaboration (HIV-NAT), Bangkok and the Maharaj Nakorn Chiang Mai Hospital, Chiang Mai, Thailand. The study was performed in accordance with the International Council for Harmonization E6 Guidelines for Good Clinical Practice (ICH GCP) and the Declaration of Helsinki (1964 amended in 2008). All participants provided written informed consent prior to the start of the study. Patient information is anonymous.

There were two treatment cohorts. The first cohort received 100 mg capsule of BIT225 or placebo QD with ART for 12 weeks. Cohort 2 were given two 100 mg capsules BIT225 (200 mg BIT225) or placebo QD with ART. ART was 1 fixed dose combination tablet of Atripla.

The purpose of the study was to assess the additional impact of BIT225 with ART in the treatment of HIV-1 infected subjects. One purpose was to determine the efficacy of 12 weeks of BIT225 treatment in HIV-1 infected subjects receiving ART by measuring plasma viral load decay and modelling HIV-1 decay. Another purpose was to determine the safety and tolerability of BIT225 QD administered for 12 weeks in HIV-1 infected subjects on ART. Other purposes of the study were to determine if 12 weeks of BIT225 treatment in addition to ART impacts levels of soluble CD163 (sCD1263), a primary biomarker of monocyte and macrophage immune activation, and to evaluate the pharmacokinetics (PK) of BIT225 QD administered for 12 weeks in combination with ART in subjects infected with HIV-1.

The safety of the experimental treatments was assessed by monitoring adverse events, vital signs, ECG parameters, clinical laboratory tests (haematology, clinical chemistry, coagulation and urinalysis) and physical examinations.

Blood samples were collected from the BIT225 100 mg cohort for PK analysis of BIT225 and ART up to 24 and 96 hours post-dose on Day 1 and Week 12, respectively. Blood samples were collected at regular intervals throughout the treatment period from subjects in all cohorts to monitor treatment compliance. Plasma samples were assayed by validated LC/MS/MS methods specific for the determination of BIT225 and ART.

HIV-1 plasma viral load was determined in real time by Roche COBAS TaqMan HIV-1 currently approved version (Roche Diagnostics).

Analyses for cell populations (T cells and NK cells) were performed in real time by flow cytometry (CD4⁺, CD8⁺, CD38⁺, and HLA-DR⁺ for T cells, and CD16⁺, CD45⁺ and CD56⁺ for NK cells). ELISA assays were run for sCD163 and Th17 related cytokines. Immune activation markers measured by flow cytometry were performed in real time alongside CD4⁺, CD8⁺ and CD14⁺ enumeration at HIV-NAT laboratory, Bangkok Thailand.

The macrophage and monocyte activation marker sCD163 was measured in plasma by Macro163™ ELISA (IQ Products, Groningen, The Netherlands).

Th17 cells regulate other immune cells, specifically neutrophils and macrophages, by secreting a variety of cytokines in response to pathogens. Plasma samples were analysed to determine the level of the Th17 related cytokines (IL-21, IFN-γ, IL-1p, IL-6, IL-10, IL-17A, IL-17E/IL-25, IL-17F, IL-22, and TNF-α) during the 12 week treatment period using electro-chemiluminescence MSD U-Plex Biomarker Group Th17 (Human Combo 2) Human Multiplex Assay (MesoScaleDiscovery, MD, USA).

Plasma HIV-1 RNA declined in a similar manner after ART and 200 mg BIT225 QD or placebo QD treatment commenced. The decay profiles in the BIT225 and placebo cohorts were similar over 12 weeks of study treatment (data not shown) and similar to the decline reported for ART alone. The plasma HIV-1 RNA assay does not discriminate what cell type the virus/viral RNA was released from or if the virus is infectious. Hence, the quantitative contribution of HIV-1 replication in these cells to detectable plasma viral load may not be measurable by standard methods. Investigations are ongoing to assess the impact on replication and production of infectious HIV-1 in different cell populations.

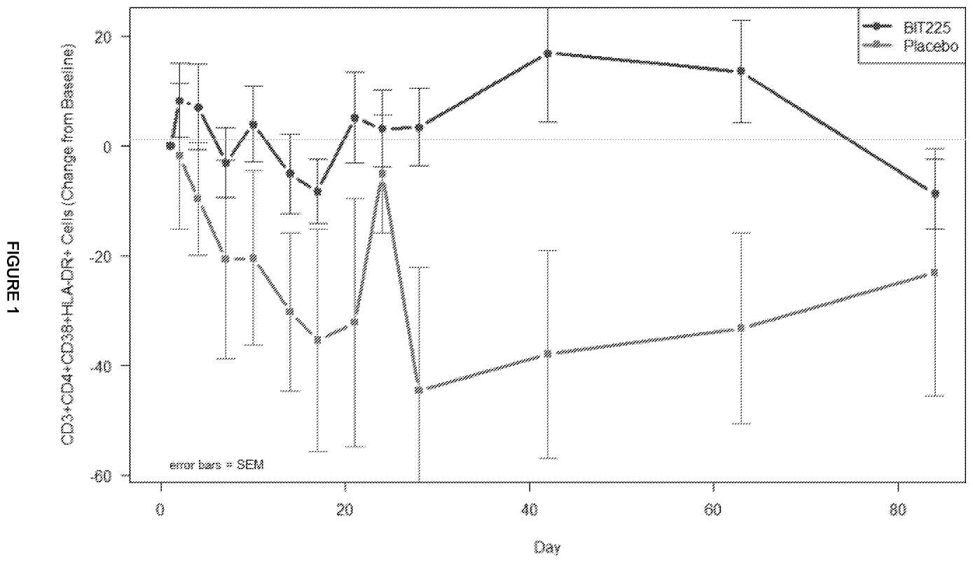

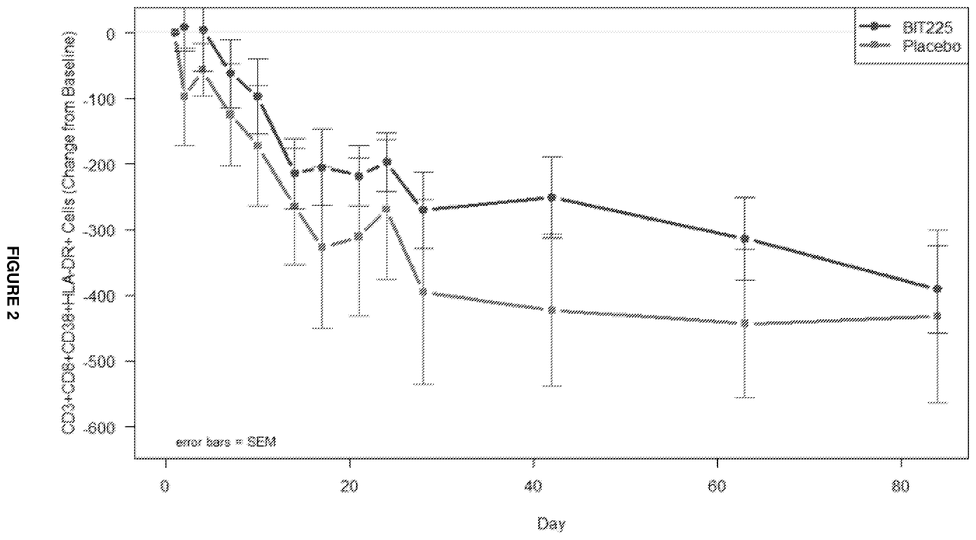

There were multiple profiles of immune cells that are not characteristic of ART that indicate that 200 mg BIT225 QD had a unique antiviral and immune modulating effect over and above ART and placebo QD. The differences are proposed to be related to the effect BIT225 has on virus replication in myeloid and lymphocytes which appear to have transiently altered innate immune recognition and function over the 12 weeks of therapy (FIG. 1 and FIG. 2). The immune cell data reported for the placebo cohort is typical of data reported for the various ART regimens, including Atripla. In FIG. 1 the number of activated CD4⁺ T cells in the placebo cohort are reduced in a similar time frame and magnitude as plasma viral load in the placebo cohort. This decay profile of activated CD4⁺ T cells is hypothesized to be related to the reduction in priming of activated CD4⁺ T cells due to the ART-induced fall in HIV-1 virion production and related reduction in presentation of viral antigens or viral-infected cells to the immune system. In contrast, the number of activated CD4⁺ cells in the 100 (data not shown) and 200 mg BIT225 cohort remained elevated until approximately 50 days before declining. This effect may be related to a change in the expression or degradation of cellular/viral factors that are involved in masking viral infected cells from the host's immune system by Vpu-mediated mechanisms.

The data in FIG. 2 shows an immediate rapid decline in CD8⁺ T cells after initiation of ART in the placebo cohort. A decline is also seen in the BIT225 cohort, but its initiation is delayed by 4 to 7 days. Over the 12-week treatment period the BIT225 cohort has more activated CD8⁺ T cells than the placebo cohort by an average of 85 cells/ml (±29; SEM; P<0.01). These estimates for the cohort difference were obtained from a two-factor linear model (factors: Day of measurement & Cohort).

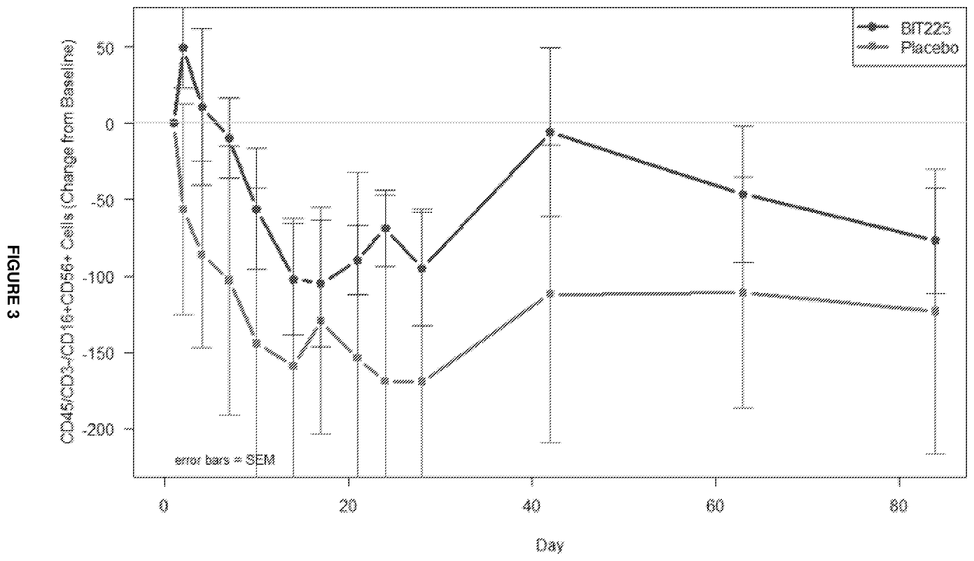

The data in FIG. 3 shows that NK cell numbers rapidly increased and peaked within 24-48 hours of commencing BIT225 and ART treatment, contrasting with an immediate decline in the ART+placebo cohort. Over the 12-week treatment period the number of NK cells was significantly increased in the BIT225 cohort compared to placebo (71±23 cells/ml; P<0.01; two factor linear model. The general decline in NK cells mirrors the CD8⁺ T cell and pVL declines. Of significance is that the NK cell numbers remain higher during BIT225 treatment which implies that HIV-1 related defects in NK signalling may be at least partially reversed by BIT225 treatment.

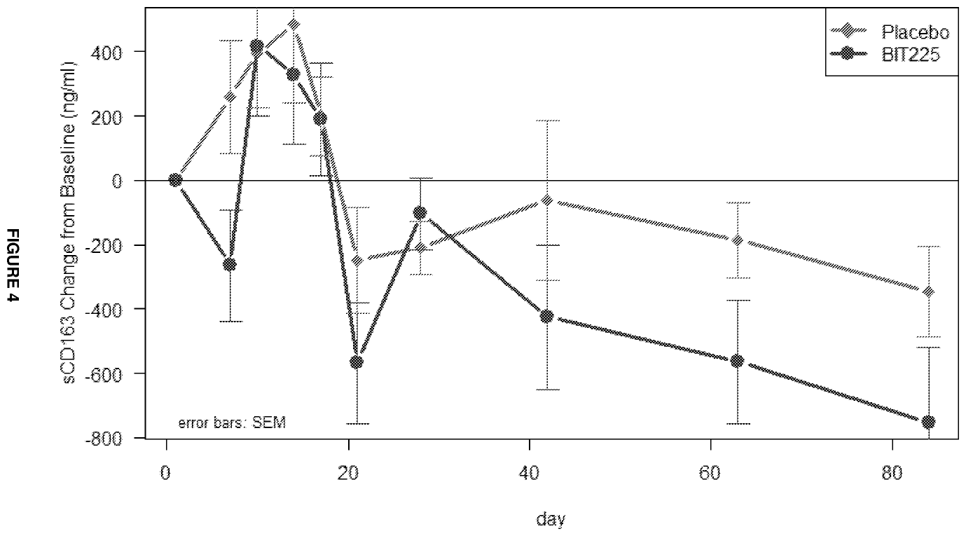

The production of sCD163 over 12 weeks of treatment was significantly reduced in the BIT225 cohort compared to ART alone (FIG. 4).

To determine whether BIT225 treatment impacted on Th17 cell levels or function several cytokines that are related to Th17 immune function were examined over the 12 Week treatment period.

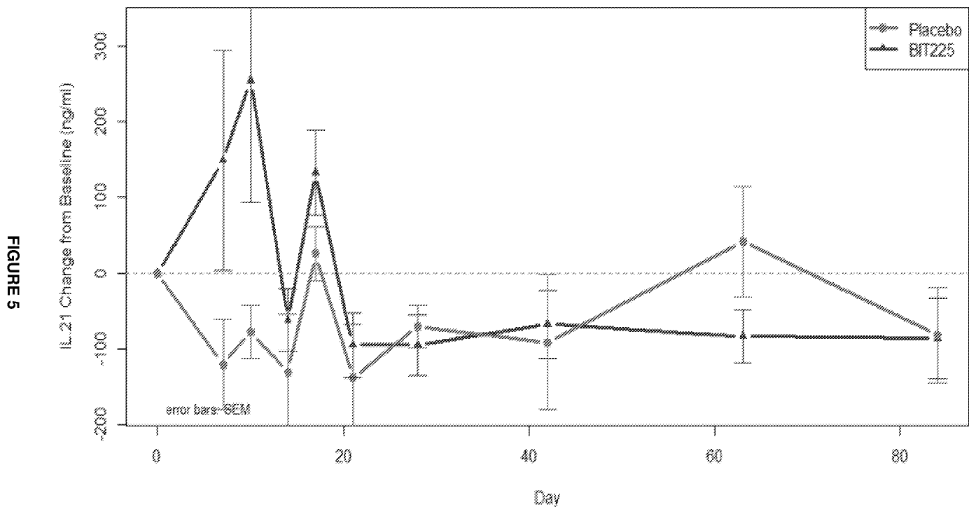

There was a statistically significant increase by ANOVA in IL-21 levels in the BIT225 cohort over the first 3 weeks of treatment (FIG. 5). Levels of IL-21 reduced after the first three weeks to levels similar to the placebo cohort and pre-treatment levels. IL-21 can be produced by follicular helper CD4 T cells (Tfh), Th17 and NK cells and this assay does not allow the assignment of IL-21 production from each of these immune cell population.

The BIT225 cohort mean for IFN-γ, an activator of macrophages and inducer of MHC class 2 expression, had a transient spike at Day 14 before returning to similar levels detected at pre-treatment. This response was detected in 16 of 18 subjects in the BIT225 cohort (data not shown). Minor fluctuations in IFN-γ levels in the placebo cohort were observed in 7 of 9 placebo subjects over the 12-week treatment period (data not shown)

The most commonly observed response for plasma IL-6 was a transient spike in first 3 weeks of treatment in 8 of 18 BIT225 subjects compared to 1 of 9 placebo subjects. The IL-6 response was reduced after 3 weeks in the BIT225 cohort but remained higher than placebo when the data was calculated as change from baseline (Log₁₀) (data not shown).

IL-10 is an anti-inflammatory cytokine that modulates the immune response. It is produced by macrophages, dendritic cells, B cells, CD4⁺ and CD8⁺ T cells. During infection it inhibits the activity of Th1 cells, NK cells and macrophages. While there was a cohort trend for a peak increase in IL-10 in the first 3 weeks followed by a rapid and sustained decline in the BIT225 cohort compared to the placebo cohort, the levels were not statistically significant over the 12-week treatment period (data not shown).

There was no difference between cohorts for production of IL-17F which increased over time in both cohorts to levels approximately double their pre-treatment (data not shown).

There were no statistical differences between BIT225 or placebo treatment cohorts for TNF-α, an inflammatory cytokine produced by macrophages and monocytes or for IL-1 B production which influences Th1 cells differentiation (data not shown).

There were no reportable results for IL-17A, IL-17F/IL-25 production as most data points were below LLQ for the assay.

BIT225 was well tolerated in both 100 and 200 mg cohorts. All subjects (36/36) experienced at least one adverse event (AE), the majority of which were mild in severity and resolved during the treatment period. Most frequently occurring AEs were similar in BIT225 and placebo treated subjects and included dizziness, nausea, headache, pyrexia and vomiting. Two subjects in the BIT225 100 mg cohort discontinued the study early due to AEs. On Day 7, one subject experienced mild sinus tachycardia, considered possibly related to Atripla and/or BIT225. A second subject was discontinued on Day 17 after reporting mild QTcB prolongation (QTcB>480 msec), considered by the Investigator not related to BIT225 or Atripla. Grade 3 dizziness was reported in 1 subject receiving 200 mg BIT225 on Day 4, deemed by the Investigator to be definitely related to Atripla and not related to BIT225. Atripla treatment was discontinued and the ART regimen changed for this subject. Two additional subjects discontinued Atripla in the first few weeks of treatment due to intolerance (dizziness) to Efaviranz; one 200 mg BIT225 and one placebo subject. These subjects were also changed to a new ART regimen and completed the treatment and follow-up period of the trial. There were no serious AEs (SAEs) or deaths.

Data from the intensive PK sampling indicated that BIT225 and Atripla achieved plasma exposures within the expected timeframes and concentration ranges. BIT225 did not significantly affect the PK characteristics of Atripla (results not shown).

The study is the first report of the safety and efficacy of BIT225, a Vpu inhibitor, in combination with ART in treatment-naive subjects with chronic HIV-1 infection. BIT225 was considered safe and well tolerated, and had no significant impact on the PK characteristics of the ART. The study shows that the addition of BIT225 to ART induced significant changes to the host immune response to HIV-1 in the background of plummeting viral load. The immunological effects were sustained for multiple weeks despite viral load reduction by several logs in the plasma within days of ART commencement.

The study showed that commencement of ART resulted in a rapid decline of activated CD4⁺ T cells in a similar profile to the viral load decline. Surprisingly, the addition of BIT225 to ART had a different effect on the profile of the decline of activated CD4 T cells, with the BIT225 cohort having a significant delay of approximately 50 days in the reduction of activated CD4⁺ T cells. This significant lag in the decline of activated CD4⁺ T cell activity indicates that the host immune system was stimulated by a mechanism not previously identified by any other ART combination.

The combination of BIT225 and ART in the current study resulted in the statistically significant decline of the macrophage activation marker sCD163 over 12 weeks of therapy to normal levels. CD163 is a membrane protein that is expressed on peripheral monocytes and macrophages, which play a central role in host response to infection and tissue damage and are important to pathogenesis of disease. Soluble CD163 is created by cleaving the CD163 receptor from monocytes and macrophage when the immune system is activated. This marker is strongly correlated with macrophage-mediated pathogenesis and is considered a better predictor than T cell activation markers of all-cause morbidity and mortality in HIV-1 patients who are on ART, in whom immune activation and inflammation are known despite ART viral control (Burdo et al., J Infect Dis 2011, 204: 1227-36; Burdo et al., J Infect Dis 2011, 204: 154-63; Burdo et al., AIDS 2013, 27: 1387-95; Knudsen et al., J Infect Dis 2016, 214: 1198-204). HIV-1 infected individuals typically have higher levels of sCD163 than age-matched HIV-negative individuals (Martin et al., PLoS One 2013, 8: e55279). ART has been shown to reduce sCD163 levels but not to the level of age-matched HIV-negative individuals (Burdo et al., J Infect Dis 2011, 204: 154-63). Increased sCD163 levels in HIV-1 infected individuals have been associated with the physical shortening of telomeres in macrophages (Srinivasa et al., J Acquir Immune Defic Syndr 2014, 67: 414-8), supporting the hypothesis that immune-aging is not reversible and early ART intervention is beneficial. Elevated sCD163 levels have been associated with unfavourable clinical outcomes of non-infectious comorbidities including cardiovascular, liver disease, type II diabetes mellitus, and cognitive decline in HIV-1 individuals (Paiardini & Muller-Trutwin, Immunol Rev 2013, 254: 78-101). The mechanism for BIT225 related sCD163 reduction to levels lower than ART alone could be related to reduction in HIV-1 replication in cells of the myeloid lineage directly, or indirectly by reversal of Vpu-related innate immune suppression. The sCD163 results suggest that BIT225 therapy could potentially result in improved health outcomes for people on long term ART.

The data from the study describes a new paradigm where the potent antiviral activity of ART is enhanced by the improved immune functions of BIT225 treatment. The most sustained immune cell effect over the 12 weeks of treatment was the statistically significant delayed reduction in activated CD4⁺ T cells numbers compared to baseline in the BIT225 cohort compared to placebo and ART. The effect was sustained for up to 50 days. The modification to the activated CD4⁺ T cell profile may be related to a dramatic change to the host's detection and response to HIV-1 infected cells and to the recovery of function of IL-21 producing cells (Th17 cells, Tfh cells and NK cells). There was a statistically significantly increased in plasma IL-21 in the first 3 weeks of BIT225 treatment. IL-21 is produced by Th17 cells to promote their differentiation and function. Th17 cells are an important subset of immune cells that are critical to the pathogenesis of HIV-1 infection. Recent studies have identified that Th17 cells are preferentially depleted in the first six months of HIV-1 infections (Klatt & Benchley, Curr Opin HIV AIDS 2010, 5(2): 135-40). This data is significant as it describes enhanced recovery of a cell type preferentially decimated by HIV-1 infection. The selective destruction of Th17 cells in the gut by HIV-1 infection prevents homeostasis of the gut epithelial barrier and is responsible for microbial translocation in to the blood (Klatt & Benchley, Curr Opin HIV AIDS 2010, 5(2): 135-40). This creates an environment of constant immune presentation, recognition and destruction of pathogen material and leads to the chronic inflammation state. High levels of proinflammatory cytokines over sustained periods of time lead to premature immune-aging and damage to multiple organs. While ART alone leads to a recovery in activated CD4⁺ T cells, this particular subset of Th17 cells does not preferentially recover to normal levels (Planas et al., Curr Opin HIV AIDS 2019, 14: 85-92). The data presented here suggests that BIT225 selectively improves the fate and activity of this cell lineage and that the addition of BIT225 to ART potentially could result in sustained clinical benefit and halt or reverse clinical immune action related organ decline.

The general overall decline in activated CD8⁺ cells in both ART cohorts (i.e. with both BIT225 and placebo) is typical of what is generally reported for ART and likely reflects diminishing HIV-1 products that stimulate the immune system. In contrast, the observed delay in the decline of activated CD8⁺ T cells in the BIT225⁺ ART cohort is not generally reported. This may be related to better antigen presentation by antigen presenting cells (APCs) and T cell signalling.

The rapid increase in NK cells numbers, which peaked within 24-48 hours of commencing BIT225 and ART, was in contrast to the immediate decline in NK cells numbers in the ART and placebo cohort. The NK cell numbers remained higher throughout the 12-week treatment with BIT225 which implies that HIV-1-related defects in NK signalling may be at least partially reversed by BIT225. The data supports the hypothesis that BIT225 reverses the down modulating effects of Vpu in NK cells and increases key cell surface receptors for efficient NK signalling and degranulation.

The initial peak in NK cells and activated CD8⁺ T cells observed in the first week of treatment with BIT225 was followed by an increase in plasma IL-21 levels which peaked at week 3. This early peak in NK and CD8⁺ T cells indicates that a new or enhanced source of antigen was detected and responded to despite the massive reduction in plasma viral antigen driven by ART. The initial peak in plasma levels of IL-21 suggests that the host immune system was driving the recovery, differentiation and function of Th17 cells, Tfh and/or NK cells. This effect was reinforced by the sustained delayed decrease in activated CD4⁺ T cells. These activated CD4⁺ T cells have the potential to recognise and destroy HIV-1 infected cells regardless of lineage. While there is some debate over the composition of cells involved in the reservoir it potentially includes hematopoietic stem cells, myeloid and T cell lineages that are previously poorly penetrated by ART alone (Hong & Mellors, Curr Opin HIV AIDS 2015, 10: 43-8; Honeycutt et al., J Clin Invest 2016, 126: 1353-66). A modest transient increase in IL-10 in the first few weeks may support the lowering of viral setpoint assumption as it has been positively associated with this outcome (Katsikis et al., PLoS Pathog 2011, 7: e1002055).

The addition of BIT225 to ART resulted in unique immune modulation not previously reported by any direct acting antiviral agent. This study provides evidence that BIT225 potentially has multiple innate immune modulating effects that may have utility in improving HIV-1 induced chronic immune activation outcomes and aid in future eradication strategies. BIT225 has a potential role as a component of a curative strategy through its activation of the host's immune response to eliminate HIV-1 infected cells that are not eliminated by ART therapy, such as cells in sanctuary sites or reservoirs (which are havens for virus latency and low-level viral replication). One possible mechanism for the immune modulation effect of BIT225 is by inducing changes in Vpu-mediated mechanisms that are involved in masking HIV-1 infected cells from the host's immune system.

### Example 3-Assays to Investigate Bit225-Specific Immunomodulatory Effects

As shown in Example 2, the addition of BIT225 to ART resulted in elevated T cell activation in the Phase 2 clinical trial compared to ART alone. The following in vitro study investigated if there was a Vpu-dependent mechanism that results in better activation and function of T cells during ART. The costimulatory receptor of T cell activation is CD28 and its counterparts are CD80 and CD86 on macrophages, dendritic cells and B cells. Experiments were designed to identify the role of accessory proteins Vpu and Nef expression on T cell activation, CD28 and CCR7 expression on CD4⁺ T cells, and CD80 and CD86 expression on monocyte-derived macrophages (MDM).

Method of virus stock generation is as described by Bobardt et al., PNAS, 2008; 105(14):5525-5530. Briefly, 293T cells were transfected with proviral plasmid (9 μg) and vesicular stomatitis virus G (VSV-G) envelope plasmid (1 μg). At day 2, VSV-G pseudotyped viruses (5 μg of p24) were harvested and used to acutely infect Jurkat cells (2×106 cells). Two days after infection, viruses were harvested, and p24 content was measured by ELISA (PerkinElmer). Viral infections were conducted in triplicate with 10 or 100 ng of p24 derived from transfected 293T cells. The following proviral constructs were used: wild-type HIV-1 pNL4.3 CCR4(X4) tropic[WT], pNL4.3 ANef, pNL4.3 AVpu, pNL4.3 ANefAVpu, and HIV-1 pBaL-1 CCR5(R5) tropic[WT].

Blood-derived CD4⁺ T lymphocytes and MDM were isolated as described in Saphire et al., J Virol 2001, 75:9187-9200. CD4⁺ T cells and monocytes were extracted from the blood of three donors that were CCR5-Delta32 negative. For generating primary human macrophages, monocytes were purified from human PBMC by negative selection (Dynal Biotech) and activated and cultured at a cell concentration of 106/ml in DMEM, supplemented with 10% FCS (HyClone), MEM amino acids, L-glutamine, MEM vitamins, sodium pyruvate (Invitrogen), and penicillin (100 units/ml), streptomycin (100 mg/ml), and 50 ng/ml recombinant human granulocyte-macrophage colony-stimulating factor (GM-CSF) (R&D Systems) and maintained at 37° C. in a humidified atmosphere supplemented with 5% CO2. To obtain MDM, cells were allowed to adhere to plastic and cultured for 6 days to allow differentiation prior to infection. Infection was conducted with 10 or 100 ng of p24 per 106 cells.

CD4⁺ T cell isolation as previously described by Geijtenbeek et al., Cell, 2000, 100:587-597. Human PBMC were purified from fresh blood by banding on Ficoll-Hypaque (30 min, 800 g, 25° C.; Amersham Biosciences). Primary human CD4⁺ T cells were purified from PBMC by positive selection with anti-CD4 Dynabeads and subsequent release using Detachabead (Dynal Biotech). Cells were cultured in RPMI medium 1640 (Invitrogen) supplemented with 10% FCS (HyClone), MEM amino acids, L-glutamine, MEM vitamins, sodium pyruvate (Invitrogen), and penicillin plus streptomycin (Cellgro) and were subsequently activated with bacterial superantigen staphylococcal enterotoxin B (SEB; 100 ng/ml) and mitomycin C-killed PBMC from another donor (10:1 PBMC:CD4⁺ cell ratio). Three days after stimulation, cells were split 1:2 in medium containing IL-2 (NIH AIDS Research and Reference Reagent Program; 200 units/ml final concentration). Culture were then split 1:2 every 2 days in IL-2 medium and infected with HIV at 7 days after stimulation. CD4⁺ T cells infection was conducted with 10 or 100 ng of p24 per 106 cells.

CD4⁺ T cells and MDM extracted from the blood of three donors were infected with VSVG-pseudotyped wildtype, Vpu-, Nef- and Vpu-/Nef-HIV-1_{NL4-3}.

Cells were treated with 3 μM BIT225 or DMSO control, and expression of CD28, CCR7, CD80 and CD86 measured by flow cytometry at 24, 48 and 72 hour time points.

Statistical analysis of the data was performed by ANOVA. Data were converted to “fold-change” by division with Donor- and Time-matched values from the untreated WT.NL4.3 dataset. Two-way ANOVA was used to statistically assess the within-virus difference between BIT225 treated and untreated means. A linear model (Fold-Change=β₀+β₁*Time+β₂*I_{BIT225}) was fitted to the dataset (N=18) for each virus which included measurements from three donors at three time-points (24 h, 48 h & 72 h); either treated with BIT225 or DMSO control.

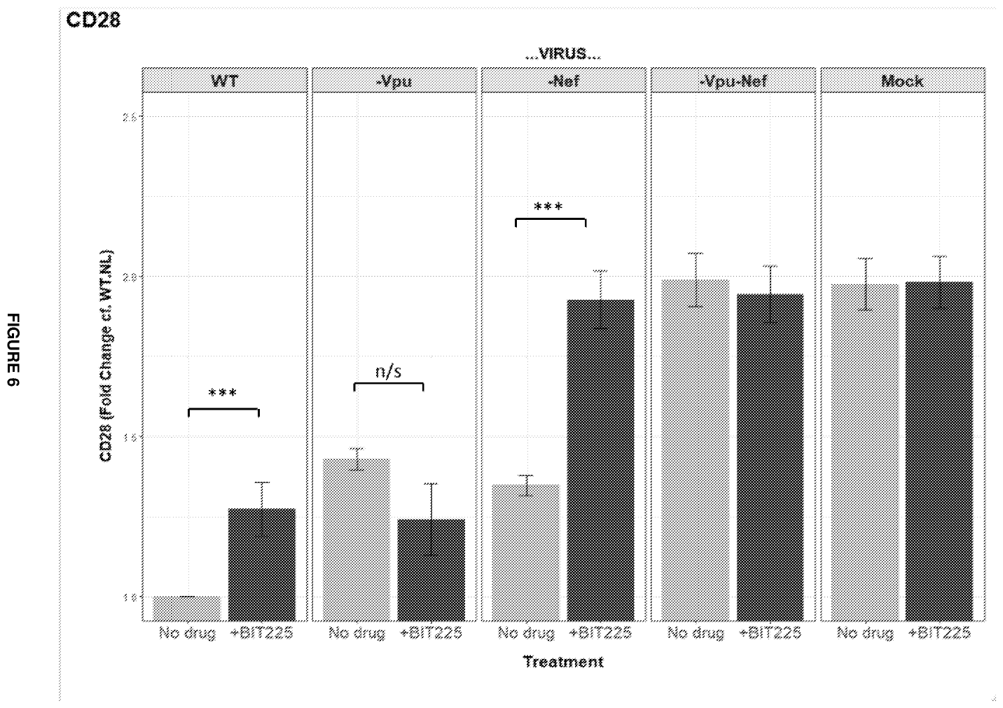

Expression of CD28 on CD4⁺ T cells was decreased by infection with wildtype (WT) HIV-1_{NIL4-3} over the 72 hour experiment (FIG. 6). The expression of CD28 was down-modulated by the expression of Vpu and Nef. BIT225 treatment of WT infected cells resulted in a partial restoration of CD28 expression that was dependent on Vpu expression only. The double deletion of Vpu and Nef resulted in restoration of CD28 expression to levels comparable to uninfected cells. BIT225 reduces Vpu-related PM CD28 downregulation as a result of HIV-1 infection. BIT225 treatment increases the expression of CD28 in a Vpu-dependent manner.

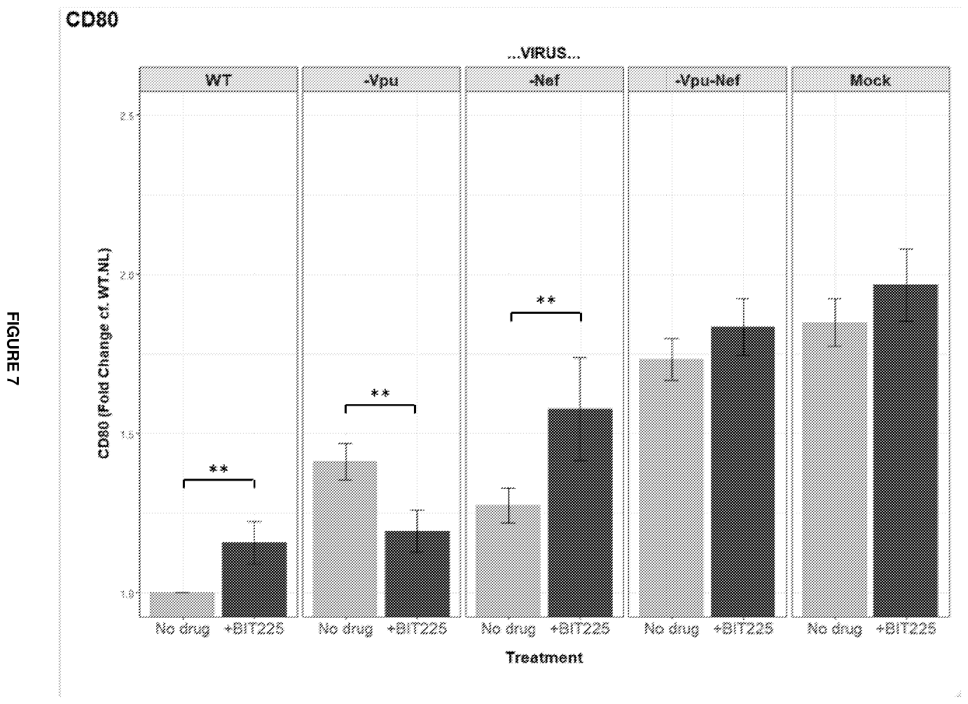

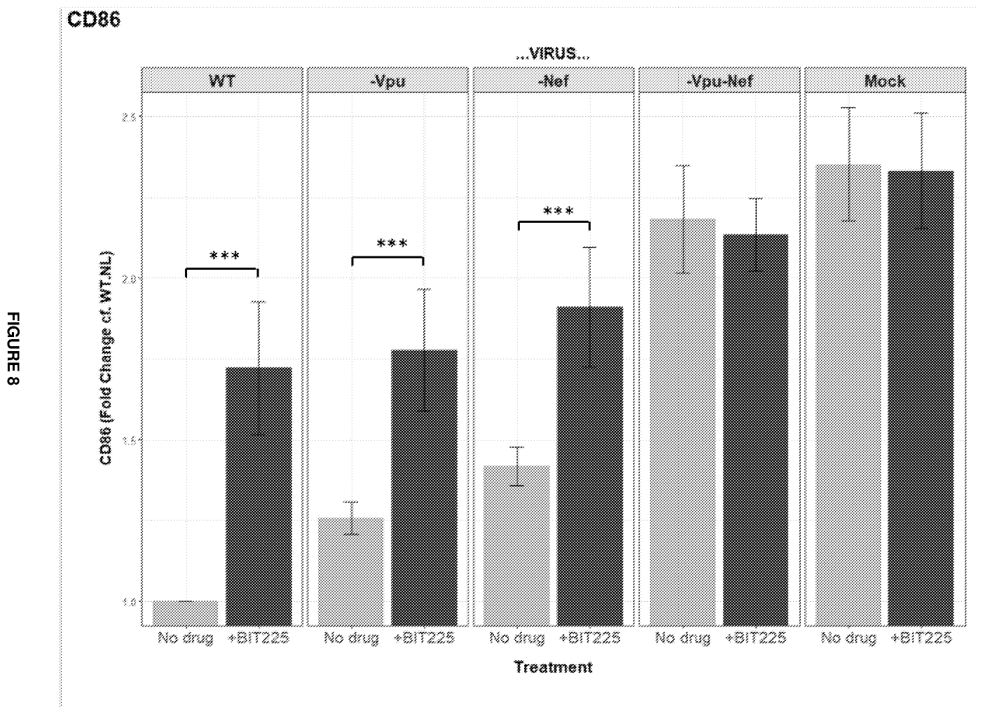

Plasma membrane expression of CD80 and CD86 were modulated by the expression of Vpu and Nef. Infection of MDM with VSVG-pseudotyped HIV-1 NL4-3 resulted in decreased plasma membrane expression of CD80 and CD86 over the 72 h experiment. Partial plasma membrane expression of CD80 and CD86 occurred in both pNL4.3 ANef and pNL4.3 ΔVpu viruses infected cells. Infection with pNL4.3 ANefAVpu did not reduce expression of either ligand, compared to mock infection (FIGS. 7 and 8). BIT225 treatment of WT or ANef virus infected cells, both of which express Vpu, resulted in a partial restoration of plasma membrane CD80 and CD86 expression. BIT225 had no observable effect on the ΔNefΔVpu HIV-1 NL4-3 double mutant virus, primarily because double deletion of Vpu and Nef returns receptors to uninfected levels.

Plasma membrane expression of CD86 on MDM was decreased by infection with WT HIV-1 NL4-3 over the 72 h experiment. The plasma membrane expression of CD86 was modulated by the expression of Vpu and Nef. BIT225 treatment of WT infected cells resulted in a partial restoration of plasma membrane CD86 expression levels. The double deletion of Vpu and Nef resulted in restoration of CD86 expression to levels comparable to uninfected cells. Unlike for the other receptors, the CD86 BIT225 treatment response was similar in the viruses with Vpu or Nef single deletions. This intriguing observation, implies a second, novel, Vpu-independent, mechanism of action by which BIT225 counteracts the Nef-dependent degradation of CD86

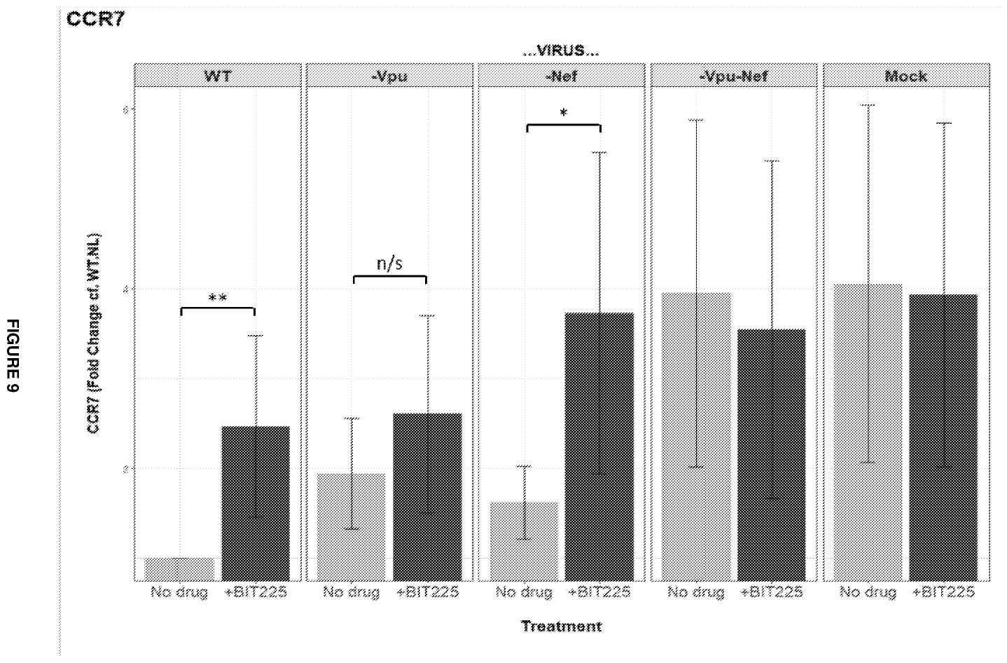

T cell function/differentiation is affected by the environment and education the cell is exposed to during its circulation through the blood and immune tissues/organs. A critical homing signal is the expression of the chemokine receptor CCR7. This study determined that CCR7 expression on CD4⁺ T cells was decreased by HIV-1 infection (FIG. 9). The expression of CCR7 was down-modulated by the expression of Vpu and Nef. BIT225 treatment of WT HIV-1 NL4-3-infected cells resulted in a partial restoration of CCR7 expression that was dependent on Vpu expression only. The double deletion of Vpu and Nef resulted in restoration of CCR7 expression to levels comparable to uninfected cells. BIT225 reduces plasma membrane CCR7 downregulation only in cells infected with viruses expressing Vpu (WT and ΔNef virus; FIG. 2B). The amount of CCR7 downregulation by Nef expression alone (ΔVpu virus) is not affected by BIT225. Together, the data confirm that the mechanism CCR7 restoration by BIT225 is dependent on expression of Vpu. This data suggest that CD4⁺ T cell function may be improved when BIT225 is added to ART.

The data suggest that BIT225 treatment can counteract Vpu-mediated downregulation of CD28 & CCR7 on CD4⁺ T cells infected with HIV-1, as well as CD80 & CD86 on MDM infected with HIV-1. The mechanism of action of BIT225 requires the presence of Vpu for modulation and restoration of CD28 and CCR7 on CD4⁺ T cells, and CD80 on MDM. However, BIT225 treatment-related enhancement of CD86 expression on MDMs infected is Vpu independent.

BIT225 appears to enhance co-stimulatory signals required for T cell activation and homing. These findings indicate novel attributes of BIT225 that suggest enhanced host immune surveillance of HIV-1. BIT225's ability to improve CCR7 plasma membrane expressions via a Vpu mechanism means that potentially more T cells will be recruited to the appropriate immunological sites for education to antigen and result in more potent immunological function to reduce or eliminate virus invasion. CCR7 and its ligands CCL19 and CCL21 provide a guidance system for immune cells to migrate to lymph nodes and contribute to both immunity and tolerance. HIV-1 accessory proteins alterations in TCR function and misguided homing signals can potentially be reasons for the continued immune activation and inflammation that drives an exhausted CD4⁺ and CD8⁺ cell phenotypes in HIV-1 infected people on long term ART. The addition of BIT225 to ART has the potential to impact on multiple cellular, chemokine and cytokine signalling pathways that are necessary for appropriate recruitment of cells to identify and eliminate virus infected cells. In Example 2, enhanced CD4 activation, seen only in the BIT225 treated group, was persistent even in the setting of rapidly falling HIV RNA. The present study suggests that BIT225 enables the detection of virus-infected cells that were neither eradicated by suppressive ART, nor readily identified by the host immune system. Accordingly, BIT225 treatment may be a valuable addition to future antiretroviral treatment, particularly for eradication of HIV-1.

1. A method for treating HIV-1 infection and regulating immune system function by lessening systemic inflammation and augmenting immune activation in a subject, comprising administering to the subject a combination comprising a) one or more antiretroviral agents and b)N-carbamimidoyl-5-(1-methylpyrazol-4-yl)naphthalene-2-carboxamide or a pharmaceutically acceptable salt thereof.

2. The method according to claim 1, wherein administration of the combination alters HIV-induced immune dysregulation.

3. The method according to claim 1, wherein the systemic inflammation is myeloid and/or monocyte inflammation.

4. The method according to claim 1, wherein administration of the combination unmasks HIV-1 infected cells.

5. The method according to claim 1, wherein the immune system is the innate immune system.

6. The method according to claim 1, wherein administration of the combination increases the number of NK cells compared to administration of the one or more antiretroviral agents alone.

7. The method according to claim 1, wherein administration of the combination increases the level of IL-21 in plasma compared to administration of the one or more antiretroviral agents alone.

8. The method according to claim 1, wherein administration of the combination increases the number of CD4⁺ T cells compared to administration of the one or more antiretroviral agents alone.

9. The method according to claim 1, wherein administration of the combination increases the number of CD8⁺ T cells compared to administration of the one or more antiretroviral agents alone.

10. The method according to claim 1, wherein administration of the combination reduces the level of sCD163 in plasma compared to administration of the one or more antiretroviral agents alone.

11. The method according to claim 1, wherein administration of the combination reduces downregulation of CD28 expression on CD4⁺ T cells infected with HIV-1 compared to administration of the one or more antiretroviral agents alone.

12. The method according to claim 1, wherein administration of the combination reduces downregulation of CCR7 expression on CD4⁺ T cells infected with HIV-1 compared to administration of the one or more antiretroviral agents alone.

13. The method according to claim 1, wherein administration of the combination reduces downregulation of CD80 expression on monocyte-derived macrophages infected with HIV-1 compared to administration of the one or more antiretroviral agents alone.

14. The method according to claim 1, wherein administration of the combination reduces downregulation of CD86 expression on monocyte-derived macrophages infected with HIV-1 compared to administration of the one or more antiretroviral agents alone.

15. The method according to claim 1, wherein the one or more the antiretroviral agents comprise a non-nucleoside reverse transcriptase inhibitor (NNRTI).

16. The method according to claim 15, wherein the NNRTI is efavirenz

17. The method according to claim 1, wherein the one or more antiretroviral agents comprise a nucleoside reverse transcriptase inhibitor (NRTI).

18. The method according to claim 17, wherein the NRTI is emtricitabine or tenofovir disoproxil fumarate (tenofovir DF).

19. The method according to claim 1 wherein the one or more the antiretroviral agents comprise efavirenz, emtricitabine and tenofovir DF.

20. Use of a) one or more antiretroviral agents and b)N-carbamimidoyl-5-(1-methylpyrazol-4-yl)naphthalene-2-carboxamide or a pharmaceutically acceptable salt thereof, for the manufacture of a medicament for treating HIV-1 infection and regulating immune system function by lessening systemic inflammation and augmenting immune activation in a subject.
